(19) United States
(12) Patent Application Publication
(10) Pub. No.: US 2024/0150282 A1
(43) Pub. Date: May 9, 2024

(54) NOVEL CRYSTALLINE COMPOUND OF SIPONIMOD HEMIFUMARATE

(71) Applicant: OLON S.P.A., Rodano (MI) (IT)

(72) Inventors: Matteo VALLI, Rodano (MI) (IT); Alberto MAZZA, Rodano (MI) (IT); Lazzaro FELICIANI, Rodano (MI) (IT); Mara SADA, Rodano (MI) (IT); Giorgio BERTOLINI, Rodano (MI) (IT)

(73) Assignee: OLON S.P.A., Rodano (MI) (IT)

(21) Appl. No.: 18/273,813

(22) PCT Filed: Jul. 7, 2021
(86) PCT No.: PCT/EP2021/068840

§ 371 Date: Jul. 24, 2023

(30) Foreign Application Priority Data
Mar. 26, 2021 (IT) 102021000007568

(51) Int. Cl. C07D 205/04

(52) U.S. Cl. CPC C07D 205/04

(57) ABSTRACT

The present invention relates to a novel crystalline compound of Siponimod Hemifumarate, to processes and to intermediates for its preparation, to pharmaceutical compositions containing it and to the use in therapy.

### SUMMARY OF THE INVENTION

The present invention relates to a novel crystalline compound of Siponimod Hemifumarate, to processes and to intermediates for its preparation, to pharmaceutical compositions containing it and to the use in therapy.

### BACKGROUND ART

Siponimod is the international common designation of 1-({4-[(1E)-1-({[4-cyclohexyl-3-(trifluoromethyl)phenyl]methoxy}imino)ethyl]-2-ethylphenyl}methyl)azetidin-3-carboxylic acid having the following formula:

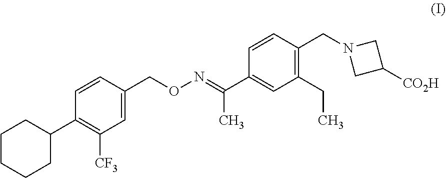 [SMILES: CCc1cc(/C(C)=N/OCc2ccc(C3CCCCC3)c(C(F)(F)F)c2)ccc1CN1CC(C(=O)O)C1]

Siponimod is the active ingredient in Mayzent® in which it is present in the form of a hemifumarate salt, a drug developed by Novartis and approved in several countries for the treatment of multiple sclerosis.

Siponimod and its synthetic route have been first described and claimed in the Patent Application WO2004/103306 and its hemifumarate salt was described in the Patent Application WO2010/080409.

It is known that different crystalline solid forms of active ingredients may exhibit different physical-chemical properties and may offer advantages, for example in terms of solubility, stability and bioavailability. Therefore, research and isolation of novel crystalline solid forms of pharmaceutically active ingredients may lead to more reliable and effective therapies.

For this reason, the preparation of novel crystalline compounds of active ingredients is considered an important technical contribution, since these novel crystals may allow better stability, bioavailability and pharmacokinetics, limit hygroscopicity and/or facilitate galenic and industrial transformations of pharmaceutical active ingredients.

However, not all crystalline compounds exhibit the characteristics necessary for their use in therapy. In fact, the stability of the crystalline form is a very important factor for a pharmaceutical active ingredient for the purposes of its formulation and storage. The active ingredient and its pharmaceutical compositions must be stable over time without showing changes in their physical-chemical component in order not to alter the bioavailability of the active ingredient itself, for example the crystalline form must not be hygroscopic and must not undergo alterations following the conventional mechanical treatments necessary for its formulation into a pharmaceutical composition.

Another important factor is the possibility of isolating crystalline forms with a high degree of chemical purity.

Considering all these factors together, the preparation of novel crystalline compounds usable in therapy is not obvious, not at all predictable and not always possible.

### OBJECTS OF THE INVENTION

A first purpose of the invention is to provide a novel crystalline compound of Siponimod, which is stable over time and not alterable by mechanical treatments, and which also has a high degree of chemical and chemical-physical purity.

A further object of the invention is to provide processes and intermediates for the preparation of said crystalline compound.

A further object of the invention is to provide pharmaceutical compositions comprising said crystalline compound.

A further object of the invention is to provide therapeutic method comprising administering said crystalline compound and said pharmaceutical compositions containing it.

### BRIEF DESCRIPTION OF THE FIGURES

FIG. 1 shows the X-ray diffraction patterns (XRDP) of the NP04 Form of Siponimod Hemifumarate of Example 1.

FIG. 2 shows the plot of the differential scanning calorimetry (DSC) analysis of Siponimod Hemifumarate of Example 1.

FIG. 3 shows the plots of thermogravimetric analysis (TGA) of Siponimod Hemifumarate of Example 1.

FIG. 4 shows the hydrogen nuclear magnetic resonance (¹H-NMR) patterns of Siponimod Hemifumarate of Example 1.

FIG. 5 shows the polarized light microscope (PLM) images of Siponimod Hemifumarate of Example 1.

FIG. 6 shows the XRDP patterns of the NP01 Form of Siponimod Hemifumarate of Example 3.

FIG. 7 shows the XRDP patterns of the NP03 Form of Siponimod Hemifumarate of Example 4.

FIG. 8 shows the ¹⁵N NMR CP-MAS patterns of the NP04 Form of Siponimod Hemifumarate of Example 1 and of Siponimod (SIP).

### DESCRIPTION OF THE INVENTION

The Applicant has isolated a novel crystalline form of Siponimod Hemifumarate that is stable under high humidity conditions and even after mechanical treatments, such as compression and grinding.

According to one of its aspects, a subject matter of the invention is a novel crystalline form of Siponimod Hemifumarate, herein named “NP04 Form”.

According to an embodiment, the NP04 Form of Siponimod Hemifumarate is characterized by showing on XRDP analysis a characteristic peak at about 2-theta=4.70±0.2, 11.89±0.2, 14.55±0.2.

According to a preferred embodiment, the NP04 Form of Siponimod Hemifumarate is characterized by showing on XRDP analysis characteristic peaks at about 2-theta=4.70±0.2, 11.89±0.2, 13.05±0.2, 14.55±0.2, 31.12±0.2.

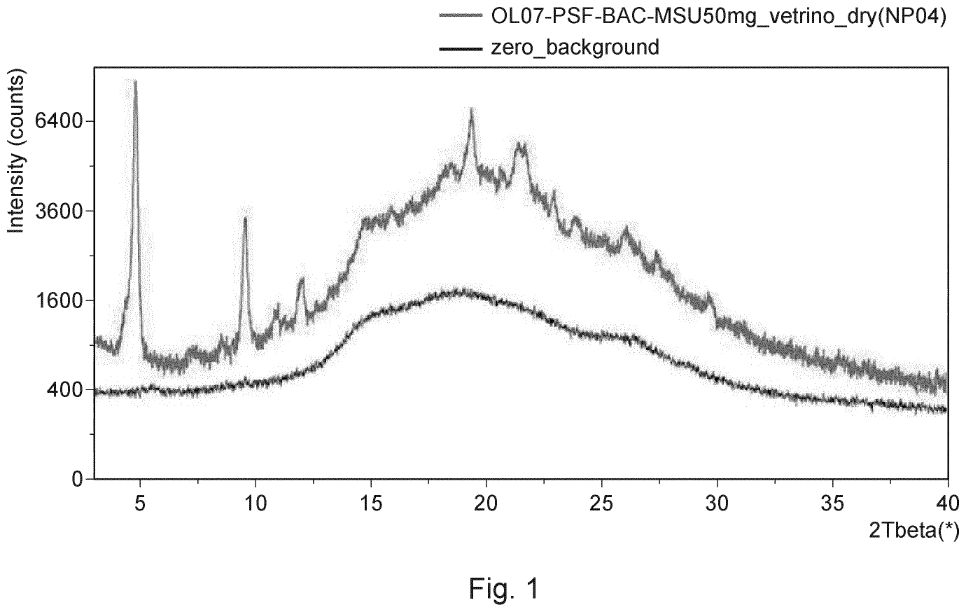

According to a preferred embodiment, the NP04 Form of Siponimod Hemifumarate is characterized by providing an XRDP patterns substantially as shown in FIG. 1.

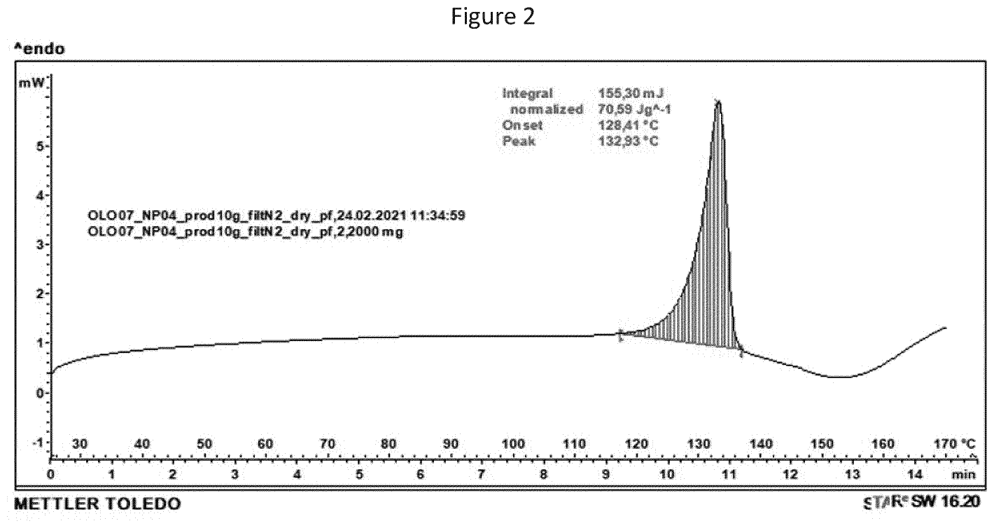

According to a preferred embodiment, the NP04 Form of Siponimod Hemifumarate is characterized by providing a DSC plot as shown in FIG. 2.

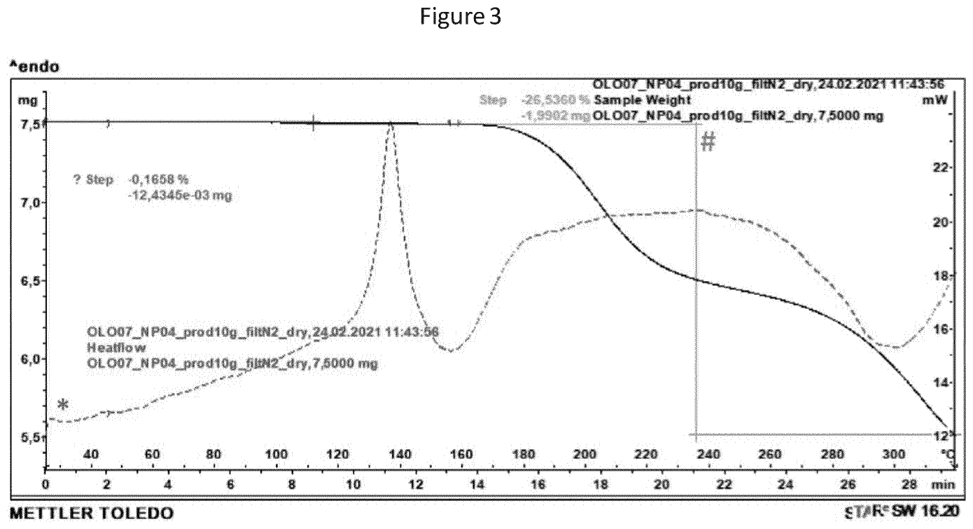

According to a preferred embodiment, the NP04 Form of Siponimod Hemifumarate is characterized by providing the TGA plot as shown in FIG. 3.

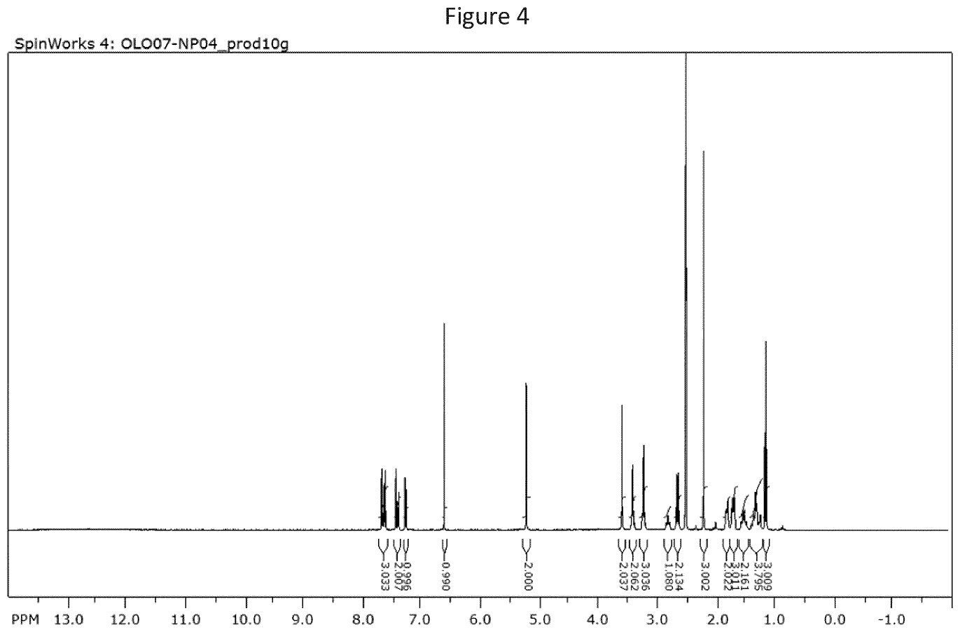

According to a preferred embodiment, the NP04 Form of Siponimod Hemifumarate is characterized by providing a ¹H-NMR patterns as shown in FIG. 4.

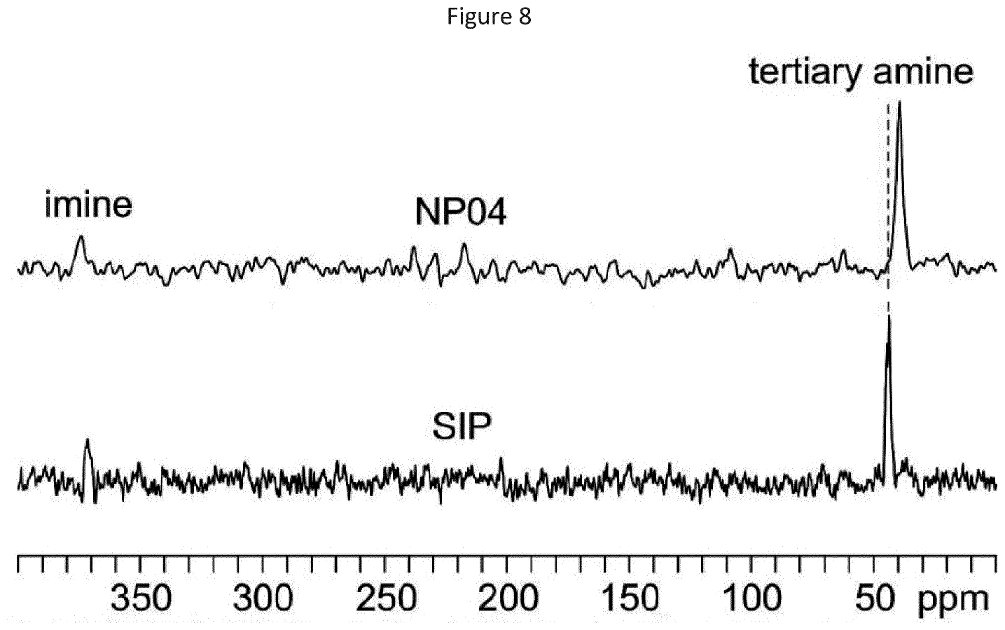

According to a preferred embodiment, the NP04 Form of Siponimod Hemifumarate is characterized by providing a ¹⁵N NMR CP-MAS patterns as shown in FIG. 8, along with the corresponding patterns of Siponimod.

According to an embodiment, the NP04 Form of Siponimod Hemifumarate is characterized by the fact to be neutral, as it can be seen from the slight shift towards lower frequencies of the aliphatic nitrogen in the ¹⁵N NMR CP-MAS patterns (shown in FIG. 8) which is not consistent with a possible protonation of the tertiary amine.

According to a preferred embodiment, the NP04 Form of Siponimod Hemifumarate is characterized by having a melting point of about 133° C., preferably 133° C.±10° C., preferably 133° C.±5° C., more preferably 133° C.±2° C.

According to another of its aspects, subject-matter of the invention is a process for the preparation of the NP04 Form of Siponimod Hemifumarate, comprising:
a) dissolving Siponimod Hemifumarate in butyl acetate by heating;
b) cooling, in the “crash-cooling” mode, the solution obtained in step (a) thus obtaining a precipitate;
c) isolating, in an inert atmosphere, the precipitate obtained in step (b); and
d) drying the precipitate obtained in step (c) in an inert atmosphere.

Siponimod Hemifumarate used as starting product in step (a) may be any crystalline or amorphous form of Siponimod Hemifumarate.

In step (a), Siponimod Hemifumarate generally melts at a temperature above 80° C., advantageously around 100° C.

In step (b), the solution obtained in step (a) is suddenly cooled, according to the “crash-cooling” technique, which provides for cooling at a rate of more than 15° C./hour, preferably more than 50° C./hour, more preferably more than 100° C./hour, even more preferably more than 200° C./hour, for example between 220 and 240° C./hour. This sudden cooling can be achieved by any possible method, for example by adding cold butyl acetate solution to the solution obtained in step (a) or by suitable cooling systems well known in the art.

According to a preferred embodiment, in step (b) it is cooled to a temperature below +10° C., preferably below +5° C., more preferably below 0° C., for example between 0° C. and −10° C.

The sudden cooling of the solution of Siponimod Hemifumarate in butyl acetate provides a precipitate that can be filtered under an inert atmosphere in step (c), according to conventional techniques.

The expression “inert atmosphere” is well known to the skilled in the art and is intended to mean, for example in atmosphere or under a flow of an inert gas such as nitrogen or argon, preferably nitrogen.

The precipitate obtained at the end of step (b) is isolated in step (c) in an inert atmosphere according to conventional methods, for example by filtration, and it is dried in inert atmosphere to provide the NP04 Form of Siponimod Hemifumarate, which has the characteristics stated above.

According to another of its aspects, subject-matter of the invention is a novel crystalline form of Siponimod Hemifumarate, herein named “NP01 Form”.

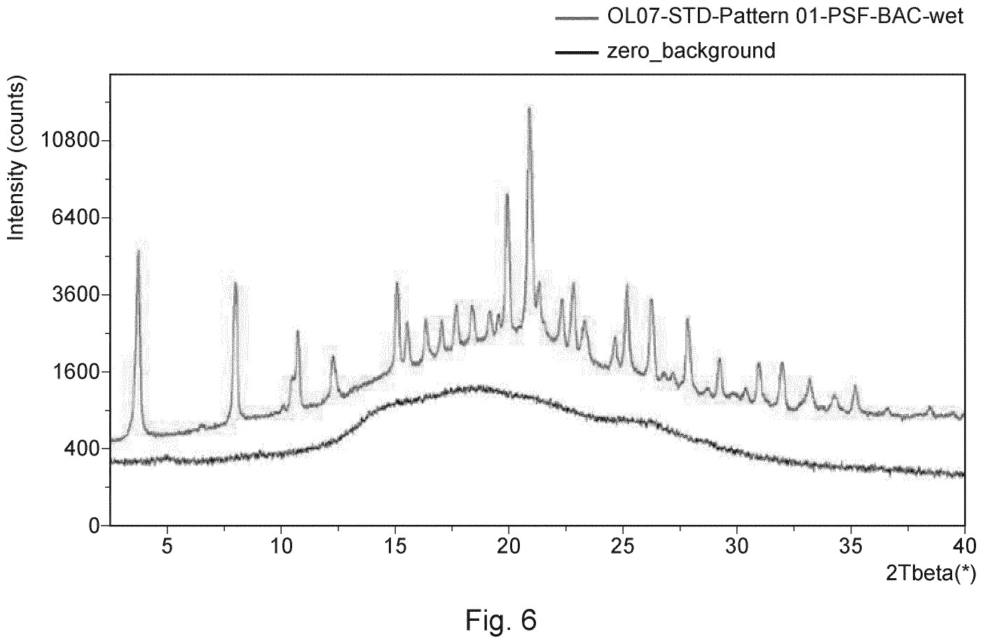

According to a preferred embodiment, the NP01 Form of Siponimod Hemifumarate is characterized by providing an XRDP patterns substantially as shown in FIG. 6.

According to another of its aspects, subject-matter of the invention is a process for the preparation of the NP01 Form of Siponimod Hemifumarate, comprising carrying out the steps (a) and (b) stated above for the preparation of the NP04 Form. It was observed that the NP01 Form described above is unstable and converts to the NP04 Form during the isolation and drying.

According to another of its aspects, subject-matter of the invention is the use of the NP01 Form of Siponimod Hemifumarate as an intermediate in the preparation of the NP04 Form.

According to another of its aspects, subject-matter of the invention is a novel crystalline form of Siponimod Hemifumarate, herein named “NP03 Form”.

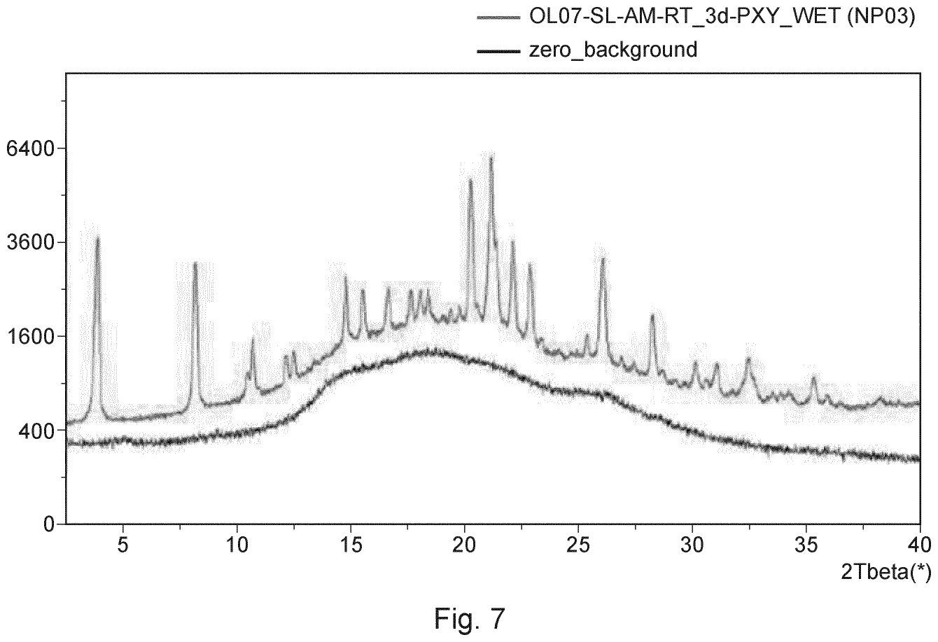

According to a preferred embodiment, the NP03 Form of Siponimod Hemifumarate is characterized by providing an XRDP patterns substantially as shown in FIG. 7.

According to another of its aspects, subject-matter of the invention is a process for the preparation of the NP03 Form of Siponimod Hemifumarate, comprising stirring a suspension of Siponimod Hemifumarate in para-xylene at ambient temperature for 24-100 hours, preferably 60-80 hours.

Thus, it is obtained the NP03 Form having the characteristics stated above.

The NP03 Form described above is unstable and converts to the NP04 Form during the isolation and drying.

According to another of its aspects, subject-matter of the invention is the use of the NP03 Form of Siponimod Hemifumarate as an intermediate in the preparation of the NP04 Form.

According to another of its aspects, subject-matter of the invention is a process for the preparation of the NP04 Form of Siponimod Hemifumarate, comprising stirring a suspension of Siponimod Hemifumarate in para-xylene at ambient temperature for 24-100 hours, preferably 60-80 hours, isolating the precipitate thus obtained, for example by filtration and drying it, according to the known methods.

The apparatuses and conditions for carrying out the analyses are set forth in the following Experimental Section.

As far as the peaks of the XRDP patterns are concerned, as it is known, it is possible that there is a minimum error in the measurement of the diffraction angle, therefore it must be understood that the peaks denoted here must be interpreted as ±0.2°.

According to another of its aspects, subject-matter of the invention is a pharmaceutical composition comprising the NP04 Form of Siponimod Hemifumarate, as defined herein, together with one or more pharmaceutically acceptable carriers. Said pharmaceutical composition may be administered via any possible route of administration and prepared according to any method known in the art.

The composition of the invention may comprise, in addition to the NP04 Form described herein, also one or more other active ingredients useful in carrying out a combination therapy.

According to a preferred embodiment, said composition is a composition for the oral administration, preferably a tablet, for example a filmed tablet. The person skilled in the art is perfectly capable of making the pharmaceutical composition of the invention according to the methods known in the art.

According to another of its aspects, it is an subject-matter of the invention a method for the treatment and/or prevention of multiple sclerosis, comprising the administration of an effective dose of the NP04 Form of Siponimod Hemifumarate to a patient in the need thereof.

According to another of its aspects, it is an subject-matter of the invention a method for the treatment and/or prevention of multiple sclerosis, comprising the administration of an effective dose of a pharmaceutical composition as described herein to a patient in the need thereof.

Said effective dose shall be established by the competent physician and shall substantially correspond to the dose already clinically used for Siponimod Hemifumarate.

As will be shown in the following Experimental Section, the NP04 Form of Siponimod Hemifumarate isolated and dried as indicated above, was shown to be stable to moisture and mechanical treatments, such as shredding and compression, and is therefore perfectly compatible with its use for the preparation of pharmaceutical compositions for the use in therapy.

On the contrary, the NP01 and NP03 Forms described herein have been shown to be unstable and constitute intermediates in the preparation of NP04.

The invention will be explained in detail in the following Experimental Section, for illustrative and non-limiting purpose.

### EXPERIMENTAL SECTION

Reactors

The reactions described in the Examples 1 to 4 were carried out by using Work station Easymax102.

X-Ray Powder Diffraction (XRDP)
Type of instrument: Rigaku MiniFlex600
Application software: Miniflex Guidance

Measurement Details
Measurement type: single scan
Sample mode: reflection

Scan
Scan interval: 3,000-40,0000 (20)
Pitch size: 0.01° (20)
Rate: 10.0°/min (20)
Scan mode: continuous

Wavelength Used
Expected wavelength type: Kα1
Kα1: 1.540598 Å
Kα2: 1.544426 Å
Kα2/Kα1 intensity ratio: 0.50
Kα: 1.541874 Å
Kα: 1.392250 Å

Instrument Details

X-Ray Generator.
Tube output voltage: 40 kV
Tube output: 15 mA
High voltage generation method: Cockcroft-Walton high frequency method
Stability: within ±0.05% for both tube voltage and tube current, with reference to ±10% of the input power variation

X-Ray Tube.
Name: Toshiba Analix Type A-26L
Anode material: Cu
Maximum power: 0.60 kW
Focus size: 1×10 mm

Kβ filter
Name: Ni-filter
Thickness (mm): 0.015
Material: Ni

Goniometer (Angle Measuring Device)
Type: vertical θ/2θ
Goniometer radius: 150 mm
Scanning axis: θ/2θ connected
2θ scanning range: +2° to +140°
Minimum pitch angle of the axis θ/2θ: 0.0050 (2θ)
Position rate: 500°/min (2θ)
Scanning rate: 0.01 to 100°/min
Reference angle: 2θ=100
X-ray “take off” angle: 6° (fixed)

Slit
DS: 1.250
IHS: 10.0 mm
SS: none (open)
RS: none (open)
Incident side Soller slit: 2.5°
Receiving side Soller slit: 2.5°

Detector
Name: 1D ultra-high rate D/teX detector
Sensing element: 1D semiconductor element
Material of the window: Be
Actual window size: 13 mm (H)×20 mm (L)
Size: 80 mm (L)

TG Analysis

The analysis has been carried out by using Mettler Toledo TGA/DSC1. The sample was weighed in a hermetically sealed aluminum dish with a perforated aluminum lid. The analysis was carried out by heating the sample from 25° C. to 320° C. at 10K/min.

Temperature Data
Temperature range: Ambient temperature . . . 1100° C.
Temperature accuracy±1 K
Temperature accuracy ±0.4 K.
Heating rate 0.02 . . . 250 K/min
Cooling time 20 min (1100 . . . 100° C.)
Sample volume ≤100 μL

Special Modes
Automation 34 sample positions
TGA-FTIR coupled to Thermo Nicolet iS10 spectrometer
Balances the XP5 data
Measuring range 5 g
Resolution 1.0 μg
Weighing accuracy 0.005%
Weighing accuracy 0.0025%
Inner ring weights 2
White curve reproducibility better than ±10 μg over the entire temperature range

DSC Analysis
The analysis has been carried out by using a Mettler Toledo DSC1 DSC. The sample was weighed in a hermetically sealed aluminum dish with a perforated aluminum lid. The analysis was carried out by heating the sample from 25° C. to 320° C. at 10K/min.

Temperature Data
Temperature range −40° C. . . . 450° C.° C.
Temperature accuracy+0.2 K.
Temperature accuracy+0.02 K
Oven temperature resolution+0.00006 K
Heating rate 0.02 . . . 300 K/min
Cooling rate 0.02 . . . 50 K/min
Cooling time 5 min (100° C. . . . 0° C.)

Calorimetric data
Sensor type FRS5
Sensor material Ceramic
Number of thermocouples 56
Time constant of the signal 1.8 s
Indium peak (height to width) 17
TAWN resolution 0.12
Sensitivity 11.9
Resolution 0.04 W
Digital resolution 16.8 million dots.

15N NMR CP-MAS
15N NMR CP-MAS spectrum was acquired with a Jeol ECZR 600 instrument, operating at 60.81 MHz, at room temperature at a spinning speed of 20 kHz, using a ramp cross-polarization pulse sequence with a 90° 1H pulse of 20 μs, a contact time of 4 or 7 ms, an optimized recycle delay between 5.2 and 14.2 s and a number of scans between 4000 and 10000. The chemical shift scale was calibrated through the signal of external standard glycine at 33.4 ppm with reference to NH3.

### Example 1

Preparation of the NP04 Form of Siponimod Hemifumarate

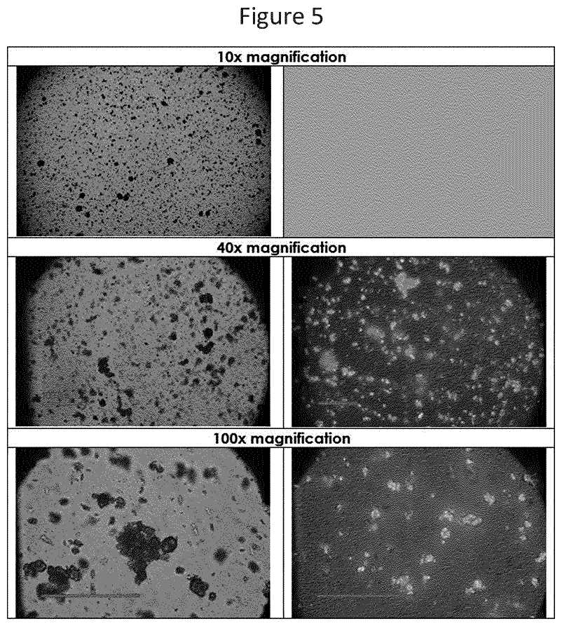

A solution of 1 g of Siponimod Hemifumarate in 100 ml of butyl acetate is prepared by heating a mixture of amorphous Siponimod Hemifumarate in butyl acetate at about 100° C. under magnetic stirring. The solution is then suddenly cooled with a cooling gradient of 3.84° C./minute to a temperature of −10° C. A white precipitate is obtained which is filtered under nitrogen atmosphere and dried still under nitrogen atmosphere. Thus, the NP04 Form of Siponimod Hemifumarate is obtained. The NP04 Form of Siponimod Hemifumarate of this Example provides the PLM images of FIG. 5 and a DSC peak at 132.56° C. as depicted in FIG. 2.

### Example 2

Preparation of the NP04 Form of Siponimod Hemifumarate

A solution of 3 g of Siponimod Hemifumarate in 100 ml of butyl acetate is prepared by heating a mixture of amorphous Siponimod Hemifumarate in butyl acetate under magnetic stirring at about 100° C. The solution is then suddenly cooled by adding −10° C. butyl acetate to the hot solution. A white precipitate is obtained which is filtered under a nitrogen atmosphere and dried. Thus, the NP04 Form of Siponimod Hemifumarate is obtained.

### Example 3

Preparation of the NP01 Form of Siponimod Hemifumarate

A solution of 1 g of Siponimod hemifumarate in 100 ml of butyl acetate is prepared by heating a mixture of amorphous Siponimod Hemifumarate in butyl acetate under magnetic stirring at about 100° C. Thus, the NP01 Form of Siponimod hemifumarate is obtained.

### Example 4

Preparation of the NP03 Form of Siponimod Hemifumarate

A suspension of 1 g of amorphous Siponimod Hemifumarate in 100 ml of paraxylene is kept under magnetic stirring for 72 hours. Thus, the NP03 Form of Siponimod Hemifumarate is obtained.

### Example 5

Stability assays of the NP04 Form

Aliquots of the compound obtained from Example 1 were subjected to the following assays:
closed in a sealed bottle for 8 days
at 25° C. with 100% relative humidity for 7 days
at 25° C. with 60% relative humidity for 7 days
at 60° C. with 75% relative humidity for 3 days
shredding treatment
Compression test under 1, 2 and 3 tons

In all cases the NP04 Form was recovered and therefore proved to be stable and unalterable by the treatments to which it was subjected.

1. A crystalline compound NP04 of Siponimod Hemifumarate of formula (I) 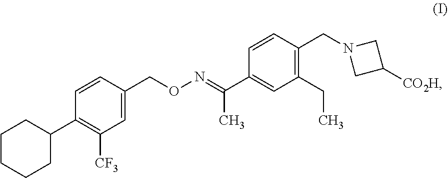 [SMILES: CCc1cc(/C(C)=N/OCc2ccc(C3CCCCC3)c(C(F)(F)F)c2)ccc1CN1CC(C(=O)O)C1]
wherein the crystalline compound has an X-ray diffraction pattern (XRDP) with a characteristic peak at 2-theta of 4.70±0.2, 11.89±0.2, or 14.55±0.2.

2. The crystalline compound according to claim 1, wherein the crystalline compound has an X-ray diffraction pattern (XRDP) with the following characteristic peaks at 2-theta of 4.70±0.2, 11.89±0.2, 13.05±0.2, 14.55±0.2, and 31.12±0.2.

3. The crystalline compound according to claim 1, wherein the crystalline compound has an X-ray diffraction pattern (XRDP) of FIG. 1.

4. The crystalline compound according to claim 1, wherein the crystalline compound has a DSC plot of FIG. 2 and/or a TGA plot of FIG. 3 and/or a ¹H-NMR pattern of FIG. 4.

5. The crystalline compound according to claim 1, wherein the crystalline compound has a melting point of 133° C.±10° C.

6. A process for preparing the crystalline compound according to claim 1, the process comprising:
a) dissolving Siponimod Hemifumarate in butyl acetate by heating to obtain a solution;
b) cooling, in the “crash-cooling” mode, the solution obtained in (a) thus obtaining a precipitate;
c) isolating, in an inert atmosphere, the precipitate obtained in (b); and
d) drying the precipitate obtained in (c) in an inert atmosphere.

7. The process according to claim 6, wherein the cooling (b) is achieved by cooling at a rate of more than 15° C./hour.

8. A method for treating and/or preventing multiple sclerosis, the method comprising:
administrating an effective dose of the crystalline compound according to claim 1 to a patient in need thereof.

9. A pharmaceutical composition, comprising:
the crystalline compound according to claim 1 and
at least one pharmaceutically acceptable carrier.

10. A crystalline compound NP01 of Siponimod Hemifumarate, wherein the crystalline compound has an X-ray diffraction pattern (XRDP) of FIG. 6.

11. A crystalline compound NP03 of Siponimod Hemifumarate, wherein the crystalline compound has an X-ray diffraction pattern (XRDP) of FIG. 7.
